(19) United States
(12) Patent Application Publication
(10) Pub. No.: US 2005/0203345 A1
(43) Pub. Date: Sep. 15, 2005

(54) Articulating paddle elevator and arthroscopic method for using same

(76) Inventors: Ken Yamaguchi, St. Louis, MO (US); Peter J. Dreyfuss, Naples, FL (US); Robert M. Weber, Chino Hills, CA (US)

Correspondence Address: DICKSTEIN SHAPIRO MORIN & OSHINSKY LLP, 2101 L Street, NW, Washington, DC, 20037, US

(21) Appl. No.: 11/070,180

(22) Filed: Mar. 3, 2005

Related U.S. Application Data
(60) Provisional application No. 60/549,147, filed on Mar. 3, 2004.

(51) Int. Cl. A61B 17/00

(52) U.S. Cl. 600/204

(57) ABSTRACT

An articulating paddle elevator for manipulating tissue during arthroscopic procedures. The articulating elevator comprises a shaft, a proximal end, and a distal end provided with an articulating paddle. The paddle may be actuated by a switch and can articulate into a standard tip for traditional manipulation of tissue, or into a rotated or articulated position. In this manner, effective manipulation and retraction of tissue from the surgical site without tissue collapse may be achieved, allowing a surgeon to better visualize the internal condition of the arthroscopic site and speed up the overall procedure.

This application claims the benefit of U.S. Provisional Application No. 60/549,147, filed Mar. 3, 2004, the entire disclosure of which is incorporated by reference herein.

### FIELD OF THE INVENTION

The present invention relates to arthroscopic surgical methods and instruments and, more specifically, to an articulating paddle elevator for arthroscopy.

### BACKGROUND OF THE INVENTION

Arthroscopic surgery involves the insertion of an arthroscope into a joint region, such as the knee, elbow or shoulder, to allow a surgeon to view the internal condition of the joint. Examples of such arthroscopic procedures are partial meniscectomies and ligament reconstructions in the knee, shoulder acromioplasties and rotator cuff debridements, and elbow synovectomies.

As a result of widening surgical indications and the development of small diameter arthroscopes, elbow arthroscopy has become increasingly used. During elbow arthroscopic surgery, a small incision is made in the skin covering the elbow so that surgical instruments may be placed in the joint and manipulated through arthroscopic visualization. A very small incision in the elbow is highly desirable as it has an obvious cosmetic advantage, low complication rates with a very low incidence of infection. A small incision in the elbow is also desirable because the elbow tissue tends to scar more easily than other tissues (due primarily to the fluid in the arm) increasing soft tissue edema.

Because only a very small incision is made during arthroscopic elbow surgery, it is often difficult to grab small regions of tissue and to subsequently apply a desired tension on the tissue within the elbow capsule, either in a direction toward or away from the arthroscopic portal. Elbow arthroscopy has an additional disadvantage in that retraction of tissue from within the elbow capsule and from the elbow bone is more difficult as the retracted tissue tends to collapse within the capsule and the retracted tissue is close to vital nerves and vascular structures.

Accordingly, a need exists for an improved surgical elbow elevator that allows controlled tissue manipulation and retraction, and improved visibility and access to the elbow capsule. A need also exists for a surgical elbow elevator that is stable during elbow arthroscopy and maintains the lifting/retracting orientation desired by the surgeon, without accidental slipping or shifting and with minimal soft tissue edema to the patient.

### SUMMARY OF THE INVENTION

The present invention overcomes disadvantages of the prior art, such as those noted above, by providing an articulating elevator having a shaft, a proximal end, and a distal end provided with an articulating paddle. The paddle may be actuated by a switch and can articulate into a standard tip for traditional manipulation of tissue, or into a rotated or articulated position.

As described below, the present invention contemplates the use of a switch mechanism (in the form of a mechanical cam) to actuate the tip of an articulating paddle elevator to a rotated or “bent tip” position. This allows the effective manipulation and retraction of tissue from the surgical site without tissue collapse, and allows a surgeon to better visualize the internal condition of the arthroscopic site and therefore speed up the overall procedure.

Other features and advantages of the present invention will become apparent from the following description of the invention, which refers to the accompanying drawings.

### BRIEF DESCRIPTION OF THE DRAWINGS

FIG. 1 is a perspective view of an articulating paddle elevator according to an embodiment of the present invention.

FIG. 2 is a front view of an articulating paddle elevator according to an embodiment of the present invention and in a non-articulating position.

FIG. 3 is a top view of the articulating paddle elevator of FIG. 2.

FIG. 4 is a front view of an articulating paddle elevator according to an embodiment of the present invention and in an articulating position.

FIG. 5(a) is an enlarged view of the distal end of the articulating paddle elevator of FIG. 2.

FIG. 5(b) is an enlarged view of the switching mechanism of the articulating paddle elevator of FIG. 2.

FIG. 6 is a partial cross-sectional view of the articulating paddle elevator of FIG. 2.

FIG. 7(a) is enlarged view of the distal end of the articulating paddle elevator of FIG. 4.

FIG. 7(b) is an enlarged view of the switching mechanism of the articulating paddle elevator of FIG. 4.

FIG. 8 is a partial cross-sectional view of the articulating paddle elevator of FIG. 4.

FIG. 9 is a lateral view of an elbow joint undergoing elbow arthroscopy and with the articulating paddle elevator of the present invention.

### DETAILED DESCRIPTION OF THE PREFERRED EMBODIMENTS

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

The term “endoscopy” encompasses arthroscopy, laparoscopy, hysteroscopy, among others, and endoscopic surgery involves the performance of surgical procedures within a patient's body through small openings as opposed to conventional open surgery through large incisions.

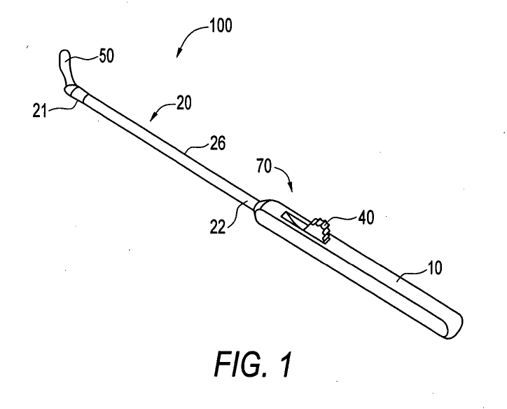

Referring now to the drawings, where like elements are designated by like reference numerals, FIGS. 1-8 illustrate an articulating paddle elevator 100 of the present invention. The articulating paddle elevator 100 of FIGS. 1-8 may be used to manipulate and palpate tissue during arthroscopic surgery such as elbow surgery, for example.

As illustrated in FIG. 1, the paddle elevator 100 includes a cannulated shaft assembly 20 provided in the shape of a cylinder or hollow tube and having a distal end 21 and a proximal end 22. The shaft assembly 20 typically has a length of about 5 cm to about 20 cm, preferably about 15 cm. The diameter of the shaft assembly is sufficiently small to facilitate introduction through access sheaths, trocars, and the like, typically being less than about 10 mm, preferably about 5 to about 7 mm.

The shaft assembly 20 includes an elongate outer tubular member 26 which houses a coaxial actuator or inner gear member 25. The actuator or inner gear member 25 has a diameter smaller than the diameter of the outer tubular member 26 and can move axially within the outer tubular member 26 of the shaft assembly 20. As described in more detail below, the distal end 21 of the shaft is configured to engage an articulating paddle, while the proximal end 22 of the shaft has a handle assembly comprising an actuator assembly designed to facilitate manual manipulation of the device and to engage and move the articulating paddle.

As illustrated in FIGS. 1-4, the shaft or body assembly 20 of the surgical instrument 100 may have a round cross-sectional shape. The shaft or body assembly 20 is formed of a rigid, medically acceptable metal or plastic material, preferably stainless steel.

In a preferred embodiment, an actuating tip or paddle 50 is located at the distal end 21 of the body assembly 20. In one embodiment, and as illustrated in FIGS. 1 and 3, for example, the actuating paddle 50 has an elliptical configuration and is used for manipulating and palpating tissue. The invention contemplates, however, other geometries and configurations for the actuating paddle 50, and is not limited to the embodiment of FIGS. 1 and 3. For example, the actuating paddle 50 may have a round or “spoon-like” configuration, among others. Preferably, the actuating tip or paddle 50 is formed of a relatively hard material to permit smooth advancement around the neurovascular structures to be protected and subsequent lifting and/or retracting of the capsular tissue.

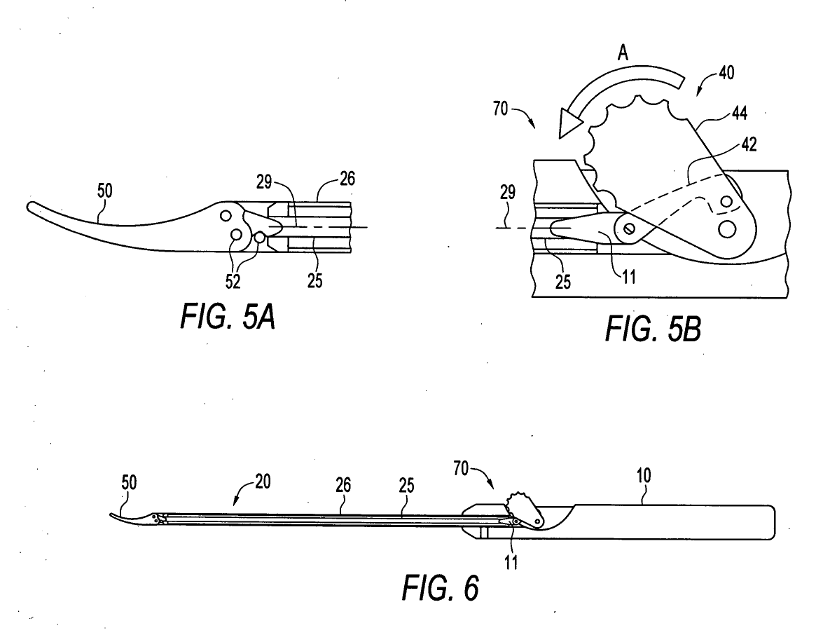

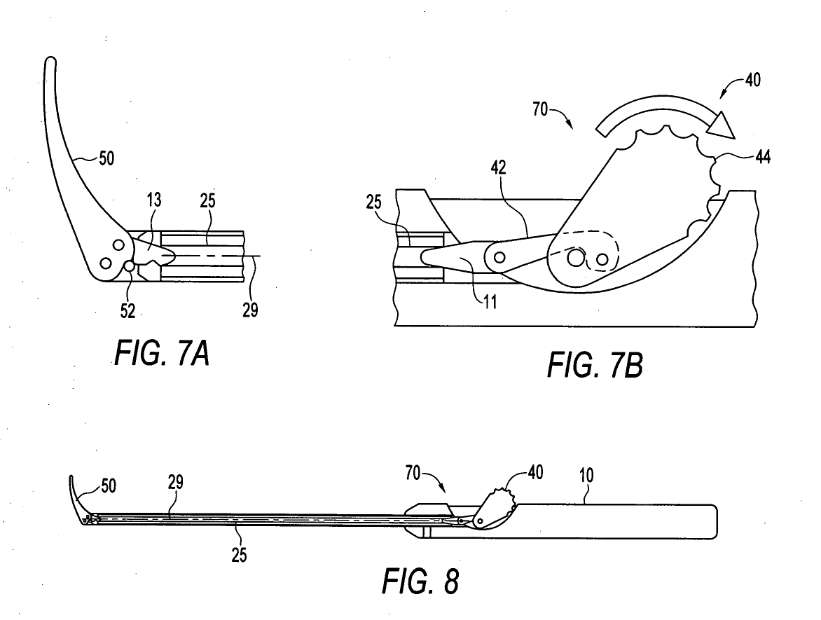

As shown in FIGS. 1-4, a handle 10 is disposed at the proximal end 22 of the shaft 20. Actuator assembly 70 comprising a switch mechanism 40 and an actuator 25 is located at the proximal end of the shaft 20 and connected to the handle 10. The switch mechanism 40 comprises a lever or thumb trigger 44, a link 42 and a cam 11. Actuator 25 of the actuator assembly 70 is connected to the link 42 and the trigger 44 of the switch mechanism 40. The switch mechanism 40 is designed to cause the actuating paddle 50 of the elbow elevator 100 to be angled relative to longitudinal axis 29 (FIGS. 5(a),5(b)), for example at about 80°, when actuated. The actuating paddle 50 is connected to actuator 25 by a plurality of stop pins 52 (FIGS. 5(a) and 7(a)).

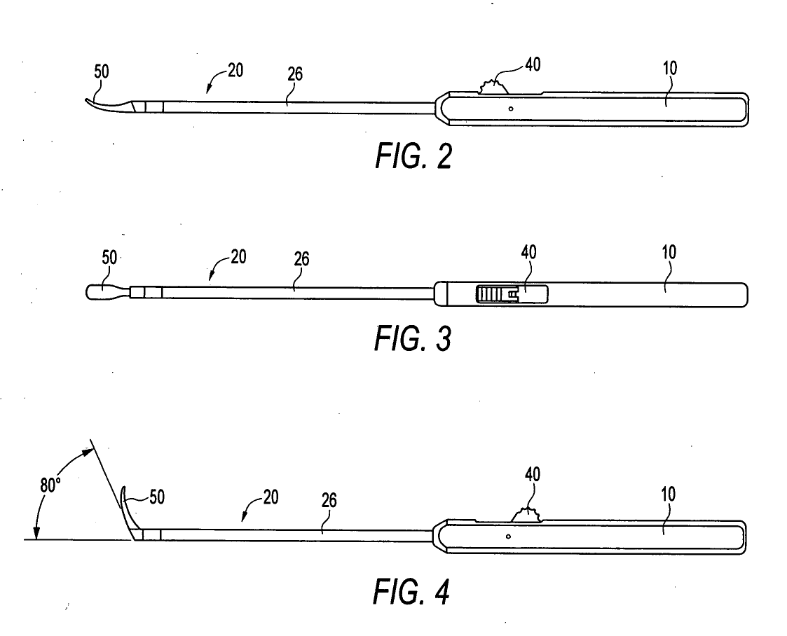

FIGS. 2-8 illustrate details of the mechanics of the articulating paddle elevator 100 of the present invention. FIGS. 2 and 3 illustrate the articulating paddle elevator 100 disposed in a first position (for example, the straight or “unlocked” position), while FIG. 4 illustrates the elbow elevator 100 disposed in a second position (for example, the articulating or “bent tip” position) and locked. FIGS. 5(a)-(b) and FIGS. 7(a)-(b) are enlarged views of the articulating paddle and switch mechanism corresponding to the straight and articulating positions of the articulating paddle elevator 100 of FIGS. 2 and 4, respectively.

The articulating paddle 50 may rotate relative to the longitudinal axis 29 of the shaft assembly 20 to an angle of about 5° to about 175°, preferably of about 10° to about 90°, and most preferably of about 20° to about 80°. For example, the embodiment shown in FIGS. 4 and 7-8 illustrates the articulating paddle 50 angled at about 80°.

As shown in FIGS. 4 and 7-8, the articulating paddle 50 is connected to the switch mechanism 40 of the actuator assembly 70 which comprises the lever or trigger 44, link 42 and cam 11. Actuator 25 is connected to the paddle 50 at the distal end. The switch mechanism 40 is mechanically connected to the paddle 50 and, when actuated, causes the paddle 50 to rotate to the 80° position shown in FIGS. 4 and 7-8. In the straight position shown in FIGS. 2 and 6, the trigger 44 is pushed in the direction of arrow A (FIG. 7(b)) and disposed in a front or “unlocked” position. This causes the actuator 25 to be biased towards the distal end of articulating paddle elevator 100.

FIG. 4 illustrates the articulating paddle elevator 100 in the “bent tip” or “locked” position, in which the articulating paddle 50 is disposed in an 80° position. To rotate the articulating paddle to the 80° position, trigger 44 is pushed backward in the direction of arrow B of FIG. 7(b). Trigger 44 includes a cam 11 which pushes the actuator 25 backward or towards the proximal end of the actuator when trigger 44 is moved backward. This mechanical action causes the paddle 50 to rotate 80° as shown.

The articulating paddle elevator 100 of the present invention described above with reference to FIGS. 1-8 may be employed in various surgical medical procedures for manipulating body tissue during surgical procedures. For example, the articulating paddle elevator 100 may be employed in endoscopic and arthroscopic procedures, including but not limited to elbow arthroscopy, shoulder arthroscopy, and other arthroscopic procedures that require manipulation (lifting and/or retracting) of tissue while maintaining capsular distention without damaging adjacent structures such as neurovascular structures, for example.

It will be appreciated, of course, that while the surgical instrument 100 may be particularly useful for performing remote procedures through access sheaths and trocars, it will also find use in open surgical procedures where its ability to manipulate tissue will also provide advantages.

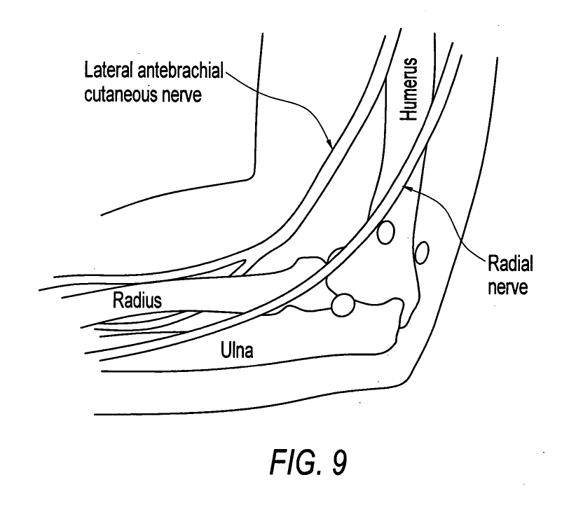

To better illustrate an exemplary surgical procedure conducted with the articulating paddle elevator 100 of the present invention, reference is now made to FIG. 9, which illustrate a side schematic view of a surgical site 90 of elbow joint 200. A surgeon advances articulating paddle elevator 100 in the “straight” or closed configuration into elbow joint 200, as shown in FIG. 9, optionally through a small cannula or portal, for example. The “straight” configuration allows the surgeon to gently insert the instrument into the elbow capsule and out of adjacent vital neurovascular structures, such as the brachial artery, the median nerve and the radial nerve, for example.

Once the articulating paddle elevator 100 is inserted into the elbow joint, the surgeon then articulates paddle 50 to a desired angle, for example to approximately 80°. The surgeon may also gradually increase or decrease the angle of the paddle (for example, from a first position to a second position), as desired and in accordance with the characteristics of the surgical site. The articulated or “bent tip” configuration allows the surgeon to lift or retract tissue out of the vision field to facilitate the surgical procedure. The articulated or “bent tip” configuration also permits the surgeon to stabilize the retracted tissue, allowing better manipulation of arthroscopic instruments within the joint. For example, by retracting tissue out of the vision field with articulating paddle 50, an arthroscopic bur or shaver 300 (FIG. 9) together with an arthroscope 350 (FIG. 9) may be employed through adjacent portals to conduct an arthroscopic procedure.

The articulated configuration of the elevator further allows the surgeon to retract the tissue away from the neurovascular structures present in the elbow joint and to maintain capsular distention, with decreased soft tissue edema and trauma to the patient.

Although the above exemplary embodiment has been described with the paddle articulating at an angle of about 80°, other angles of articulation are possible by appropriate design of cam 11. For example, a 90° angle may be used for specific locations in the elbow joint, adjacent the ulnar nerve. Although the present invention has been described in relation to particular embodiments, many other variations and modifications and other uses will become apparent to those skilled in the art.

The above description and drawings illustrate preferred embodiments which achieve the objects, features and advantages of the present invention. It is not intended that the present invention be limited to the illustrated embodiments. Any modification of the present invention which comes within the spirit and scope of the following claims should be considered part of the present invention.

1. An articulating paddle elevator, comprising:
a cannulated shaft having a distal end and a proximal end, and a longitudinal axis;
an articulating paddle located at the distal end of the cannulated shaft, the articulating paddle being capable of being moved relative to the longitudinal axis of the cannulated shaft from a first position to a second position; and
an actuator assembly located at the proximal end of the cannulated shaft and operatively connected to the articulating paddle.

2. The articulating paddle elevator of claim 1, wherein in the first position, the articulating paddle forms with the longitudinal axis of the cannulated shaft an angle of about 0°.

3. The articulating paddle elevator of claim 2, wherein in the second position, the articulating paddle forms with the longitudinal axis of the cannulated shaft an angle of about 5° to about 175°.

4. The articulating paddle elevator of claim 3, wherein the articulating paddle forms with the longitudinal axis of the cannulated shaft an angle of about 80°.

5. The articulating paddle elevator of claim 1, wherein the actuator assembly comprises an actuator operatively connected to the articulating paddle, and a switch mechanism connected to the actuator.

6. The articulating paddle elevator of claim 5, wherein the switch mechanism comprises a trigger, a link and a cam.

7. The articulating paddle elevator of claim 1, wherein the elevator is used in elbow arthroscopy.

8. The articulating paddle elevator of claim 1, wherein the actuator assembly rotates the articulating paddle relative to the longitudinal axis of the cannulated shaft.

9. The articulating paddle elevator of claim 1, wherein the articulating paddle has an elliptical configuration.

10. A surgical instrument for elbow arthroscopy, comprising:
a shaft having a proximal end, a distal end and a longitudinal axis, the shaft comprising an actuator disposed coaxially within the shaft;
a paddle located at the distal end of the shaft and coupled to the actuator, the paddle being capable of rotating relative to the longitudinal axis of the shaft; and
a switch assembly located at the proximal end of the shaft and coupled with the actuator.

11. The surgical instrument of claim 10, wherein the switch assembly comprises a cam mechanism that controls the movement of the actuator by manually pushing a trigger of the switch assembly.

12. The surgical instrument of claim 10, wherein the paddle has an eliptical configuration.

13. A method of endoscopically manipulating tissue, comprising the steps of:
providing an articulating paddle elevator comprising a cannulated shaft having a distal end and a proximal end; an articulating paddle located at the distal end of the cannulated shaft, the articulating paddle being capable of being moved relative to a longitudinal axis of the cannulated shaft from a first position to a second position; and a switch assembly located at the proximal end of the cannulated shaft and operatively connected to the articulating paddle;
positioning the articulating paddle elevator in the proximity of tissue to be manipulated at a surgical site;
advancing the articulating paddle elevator in the first position through the tissue; and
actuating the switch assembly to move the articulating paddle from the first position to the second position and to manipulate tissue at the surgical site.

14. The method of claim 13, wherein, in the first position, the articulating paddle forms with the longitudinal axis of the cannulated shaft an angle of about 0°.

15. The method of claim 13, wherein, in the second position, the articulating paddle forms with the longitudinal axis of the cannulated shaft an angle of about 5° to about 175°.

16. The method of claim 13, wherein the surgical site comprises elbow capsular tissue.
